(19) United States
(12) Patent Application Publication
(10) Pub. No.: US 2025/0144435 A1
(43) Pub. Date: May 8, 2025

(54) COLD ATMOSPHERIC PLASMA GENERATOR AND RESPIRATORY EQUIPMENT FOR THE STIMULATION OF CELLULAR REGENERATION FOR LIVING BEINGS

(71) Applicant: BIOENGINEERING FOR THE WORLD CORPORATION S.L., Las Rozas de Madrid (Madrid) (ES)

(72) Inventors: Osvaldo Daniel CORTÁZAR PERÉZ, Las Rozas de Madrid (Madrid) (ES); Pedro Luís LLANA GARCÍA, Las Rozas de Madrid (Madrid) (ES); Alejandro LOREDO FERNÁNDEZ, Las Rozas de Madrid (Madrid) (ES); Ana María MEGÍA MACÍAS, Las Rozas de Madrid (Madrid) (ES)

(73) Assignee: BIOENGINEERING FOR THE WORLD CORPORATION S.L., Las Rozas de Madrid (Madrid) (ES)

(21) Appl. No.: 18/835,915

(22) PCT Filed: Feb. 7, 2022
(86) PCT No.: PCT/ES2022/070055

§ 371 Date: Aug. 5, 2024

(51) Int. Cl. A61N 1/44; A61M 16/00; A61M 16/10; H05H 1/24

(52) U.S. Cl. CPC A61N 1/44; A61M 16/0066; A61M 16/10; A61M 16/107; H05H 1/24; H05H 2245/10; H05H 2245/30

(57) ABSTRACT

There is a cold atmospheric plasma generator configured to receive air as input substance and expel RONS, Reactive Oxygen and Nitrogen Species, suitable for being inhaled by living beings as output substance, and respiratory device that integrates said cold atmospheric plasma generator.

### CROSS-REFERENCE TO RELATED APPLICATION

The present application is a national phase application claiming priority from PCT Application No. PCT/ES2022/07055, filed Feb. 7, 2022, which is incorporated herein by reference in its entirety.

### FIELD OF THE DISCLOSURE

The present disclosure relates to a cold atmospheric plasma generator and to respiratory equipment that incorporates the cold atmospheric plasma generator.

### DESCRIPTION OF THE RELATED ART

In physics and engineering, plasma is defined as the fourth state of matter. This state is achieved by transferring enough energy to a gas so that a significant portion of its molecules become ionized. Examples of plasmas in nature are of interest, such as those of electrical discharges in storms and those of the aurora borealis or australis. In addition, there are plasmas that are used technologically, fluorescent and neon lighting tubes are a classic example along with plasma screens. Plasma is also used to cut materials as in the case of plasma torches that use its high temperature and great ability to transfer heat. However, in the last twenty years, devices capable of creating gas plasmas at low temperature atmospheric pressure have been developed, some of which are compatible with living tissue. The key for these plasmas to not damage tissues is the fact that they are out of thermodynamic equilibrium. This characteristic strongly limits the ability to transfer heat to other bodies with which they come into contact, receiving the name of cold atmospheric plasmas or CAP from its acronym. This type of plasma produces a certain concentration of activated oxygen and nitrogen molecules that are called: Reactive Oxygen and Nitrogen Species or “RONS” from its acronym. The importance of RONS is due to its disinfectant and regenerative properties on mammalian cells. This has given rise to the development of a new health branch called plasma medicine that is in full growth phase and within which this disclosure fits.

CAPs are capable of annihilating bacteria, fungi and viruses with hardly any negative effects on the healthy cells of an organism, as evidenced by the overwhelming number of international publications on the subject. There is a broad international consensus about the scientific basis behind the clinical effects observed when using CAPs. RONS have been shown to be responsible for these effects by virtue of their ability to produce oxidative stress on cells and microorganisms to which cold atmospheric plasma is applied. The difference between the tolerance levels of oxidative stress between a healthy cell and harmful microorganisms is the reason for the above-mentioned selectivity that the CAPs have. Due to structural differences between healthy cells and bacteria, fungi and viruses, such as the size and lack of protection of the DNA of the latter, the level of tolerance is of the order of a hundred times greater in favor of healthy cells. The oxidative stress produced by the RONS from cold atmospheric plasma does not damage healthy tissue, but it eliminates harmful microorganisms, including resistant bacteria.

Taking advantage of the disinfection capacity of CAPs, air purification systems have been developed that use them, such as those detailed in patents: U.S. Pat. Nos. 8,529,830 B2, 7,824,475B2, 7,427,313B2, EP2663403B1 and EP3097750B1, among others. These systems incorporate different types of plasma generators to treat the air in order to eliminate microorganisms and thus achieve a certain degree of cleanliness of the air.

A number of processes have been identified during which a low or sublethal dose of a stressful agent or stimulus is capable of activating an adaptive response that increases the resistance of a cell or organism to higher intensity stress. This response is defined as hormesis. Normally we are exposed to hormetic agents such as radiation, heat, heavy metals, antibiotics, pro-oxidant substances, or physical exercise wherein food restriction is also included. The hormetic reaction involves the expression of a large number of genes that encode cytoprotective proteins such as chaperones of the type that respond to thermal stress, antioxidant enzymes, growth factors, metallothioneins, among others. When mammalian cells, including human cells, are exposed to low doses of RONS, these change the intensity of expression of the genes involved in the hormetic response in a period of approximately 9 h. The expression of constitutive, growth and proliferative genes decreases, while the expression of protective genes increases, allowing hormetically stimulated cells to be significantly more resistant to future oxidative stress up to 15 and 30 hours after stimulation. Macroscopically, the hormetic response can be compared to an immune response to vaccination, but with the great difference that the former does not keep long-term memory (beyond 30 hours), and to once again have the hormetic effect, the cells or organisms must be re-exposed to the inducing agent.

### SUMMARY OF THE DISCLOSURE

The present disclosure relates to a respiratory device that produces and projects onto a human or animal patient a laminar air flow containing a specific concentration of reactive oxygen and nitrogen molecules for inhalation. These RONS are generated by a cold atmospheric plasma generator configured to receive air as an input substance and expel RONS as an output substance and that is part of the respiratory device and that uses the hormetic response of humans and animals particularly against very low levels of oxidative stress.

Thus, the proposed disclosure consists of a respiratory device that produces a hormetic reaction in humans and animals through the inhalation of small doses of RONS dissolved in the air that have been produced by a cold atmospheric plasma generator.

The proposed device forms a clean air generation unit activated by cold atmospheric plasma that is applied to the patient both in a closed room and in a semi-open chamber specifically designed for this purpose.

For a better conceptual understanding of the disclosure, it can be considered as composed of two main parts whose functions can be integrated and allow obtaining a laminar flow of air that is relatively rich in RONS produced by a cold atmospheric plasma generator.

Cold atmospheric plasma generator: Plasma is a state of matter wherein ionized particles and electrons move at high speeds producing high-energy and high-frequency shocks that favor the production of chemical reactions that would not occur under normal environmental conditions. This characteristic is what allows the formation of RONS from oxygen and nitrogen molecules.

Respiratory device: This device may include the cold atmospheric plasma generator and is responsible for conditioning the air before being additionally activated by cold atmospheric plasma. This device consists of a silent air blower and a HEPA (High-Efficiency Particulate Arrestance) air filter. The assembly works in such a way that microorganisms and particles are eliminated from the air, including the nanoparticles present. The set assembly includes a nozzle design, for the production of a laminar flow of air. The absence of turbulence in the laminar flow is one of the keys to being able to optimally transport the RONS in the air flow.

The present disclosure does not intend to delve further into the benefits of clean air in health and the need for purification systems, but rather opens a new field of use for RONS that generates cold atmospheric plasma for therapeutic use based on the hormesis concept.

### BRIEF DESCRIPTION OF THE DRAWINGS

To complement the description that is being made and in order to help a better understanding of the characteristics of the plasma generator and the respiratory device according to a preferred example of its practical embodiment, an integral part of said description is attached, a set of drawings wherein, by way of illustration and not limitation, the following has been represented:

FIG. 1 shows a conceptual scheme of the respiratory device that integrates the cold atmospheric plasma generator according to the present disclosure and its application to a patient.

FIG. 2 shows a cross-sectional side view of the cold atmospheric plasma generator according to the present disclosure.

FIG. 3 shows a perspective cross-sectional section of the cold atmospheric plasma generator according to the present disclosure.

FIG. 4 shows a cross-section of the respiratory device that integrates the cold atmospheric plasma generator, wherein tests and measurements of the proposed system were carried out.

FIG. 5 shows the image obtained from measuring the tension and current signals between the electrodes of the cold atmospheric plasma generator.

FIGS. 6A and 6B show two light emission spectra obtained from the cold atmospheric plasma produced by the cold atmospheric plasma generator by means of an optical fiber inserted inside the ground electrode.

### DETAILED DESCRIPTION OF THE DISCLOSURE

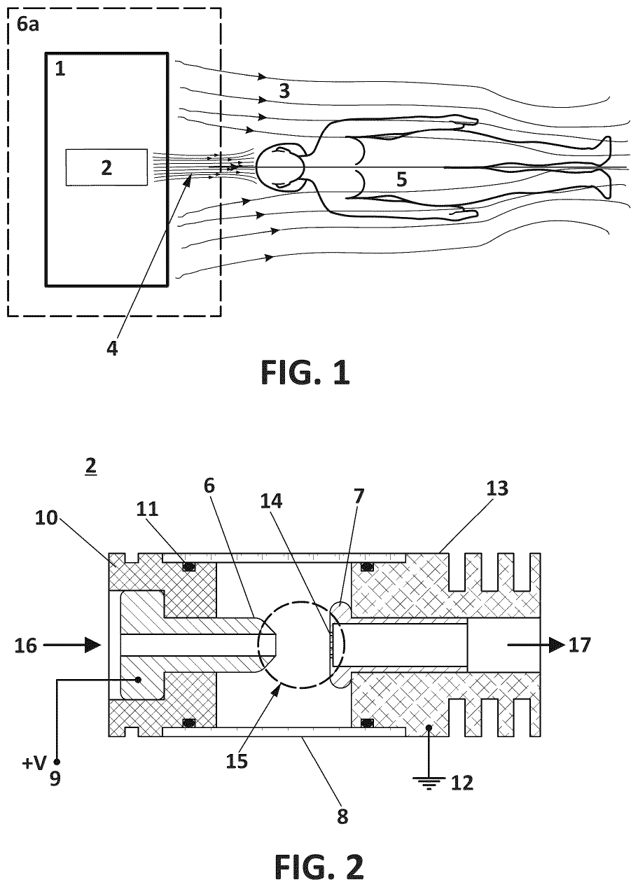

FIG. 1 shows a conceptual scheme in the form of a block diagram indicating the location of the respiratory device (1) and the cold atmospheric plasma generator (2). The respiratory device (1) is housed in a box (6a) together with the cold atmospheric plasma generator (2) that injects a plasma-activated air flow (4) into the laminar flow of clean air (3). The total and laminar flow of air is projected on the patient (5).

The laminar flow of clean air (3) is enriched by the injection of the plasma-activated air flow (4) coming from the cold atmospheric plasma generator (2). This laminar flow of air enriched with RONS generated by the cold atmospheric plasma generator (2) is projected onto the patient (5) for inhalation. The entire system is mounted inside the box (6a) and is connected to the home electricity distribution network from wherein it obtains the energy necessary for its operation.

If the air is not properly filtered and treated, it can carry polluting particles of all kinds, including those from the combustion of hydrocarbons inside the cold atmospheric plasma generator (2). In this case, unpredictable chemical reactions could take place with the polluting molecules when they come into contact with the plasma, as well as the compounds that could be generated. Therefore, the use of the previous stage of filtering the air, before being used in the cold atmospheric plasma generator (2), is recommended to allow that the reactive molecules that are generated are only oxygen and nitrogen. This allows unknown compounds not to be introduced into the airflow being applied to the patient.

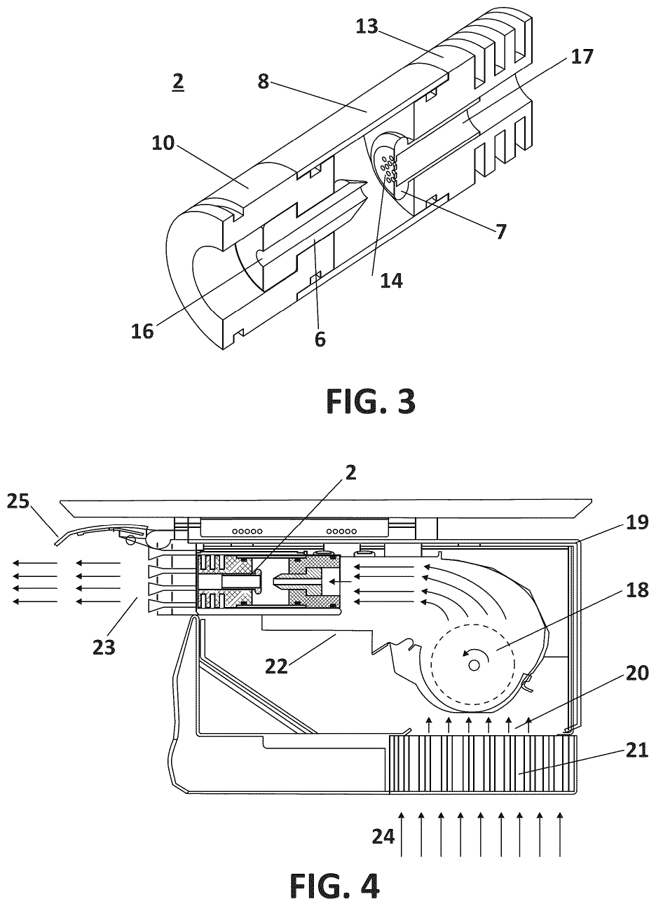

FIGS. 2 and 3 show cross sections of the cold atmospheric plasma generator (2).

The cold atmospheric plasma generator (2) is made up of two metallic electrodes (6) and (7) facing each other separated by a space (15). The frustoconical positive electrode (6) is connected to a high tension voltage source (9) and the flat metallic electrode (7) is electrically connected to ground (12). Both electrodes have cylindrical symmetry and are held in a coaxial position by two centering pieces, in particular, a supporting piece made of insulating material (10) and a metallic piece (13), respectively. Both centering pieces are kept in a coaxial position by means of a tube of insulating material (8) that is adjusted by means of O-rings (11) to them. The air inlet and outlet are references (16) and (17), respectively.

The positive electrode (6) is connected to a voltage source (9) +V and is held in place by a supporting piece of insulating material (10) that fits with the tube of insulating material (8) by means of an O-ring (11). The positive electrode (6) has a frustoconical shape and has a through hole concentric with its axis. On the other hand, a flat metallic electrode (7) is connected to ground (12) and facing the previous one held by a metallic piece (13) that closes the assembly at the other end of the tube of insulating material (8) with an O-ring. The flat ground electrode (12) has rounded edges and a non-through hole concentric to its axis. This hole or axial perforation leaves a thin wall that forms the front of the ground electrode which, in turn, has a series of through holes (14) of smaller diameter that communicate the interior of the flat metallic electrode (7) with the space (15) that separates the two electrodes as indicated in FIG. 2 with a dashed circle.

The air to be activated by means of the cold atmospheric plasma enters the system from the rear part (air inlet (16)) of the positive electrode (6) reaching the space (15) between the electrodes (6) and (7), wherein it circulates and passes through the through holes (14) penetrating inside the flat metallic electrode (7) and finally leaving the system through its outlet (17).

Once an air current is established inside the cold atmospheric plasma generator (2), the voltage is applied to the positive electrode (6) in such a way that its value is high enough to exceed the dielectric capacity of the air in the space (15) between the electrodes (6), (7). In this way a column of cold atmospheric plasma of circulating air is established between the electrodes (6) and (7). Inside the plasma column, reactive oxygen and nitrogen molecules, RONS, are formed, which are dragged by the air current towards the outside through the outlet (17) of the flat metallic electrode (7).

The cold atmospheric plasma generator (2) can inject an air current activated by atmospheric plasma (4) in the main laminar flow that is projected on the patient (5) as shown in FIG. 1. FIG. 4 shows a cross section of a diagram showing a preferred embodiment of the respiratory device (1) according to the present disclosure.

The respiratory device (1) comprises a box (19) made of a material that can be insulating or grounded metal and that contains an air impeller (18) that absorbs air (24) from the outside through an inlet opening (20) wherein an air filter (21) is housed, for example, a HEPA filter, forcing the air flow to pass through it completely. The clean air is projected to the outside through a nozzle (22) that produces a laminar flow. Some of the clean air is forced through the cold atmospheric plasma generator (2) configured to expel RONS. The main air current and the air current from the RONS generator are mixed at the outlet opening (23). A slat (25), preferably made of metallic material, allows directing the air flow to the outlet.

In this embodiment, an air impeller module (18) with cylindrical blades 30 cm long has been used, which is oriented horizontally and is housed in an airtight box made of insulating material. Said box has a rectangular inlet opening (20) oriented downwards wherein the HEPA filter is housed, whose measurements are 30 cm long by 10 cm wide with a thickness of 3 cm. A rectangular nozzle (22) made of sheet metal is installed at the outlet of the air impeller that slightly concentrates the air flow obtained and projects it horizontally forward in the form of a laminar flow air tongue. The set works in such a way that it guarantees that all the projected air has previously passed through the HEPA filter, being free of nanoparticles and other contaminants. The air impeller can operate in four speed regimes which, with the entire system assembled, translate to laminar air flow velocities of 0.40, 0.70, 0.90 and 1.25 m/s measured at 1 m away from the outlet opening (23).

Part of the clean air produced is driven through the cold atmospheric plasma generator (2) which is fixed in the center of the nozzle (22). This portion of air is incorporated into the main flow at the outlet opening (23) corresponding, in this particular case, to 5% of the total. The cross section of FIG. 4 is made in the plane wherein the cold atmospheric plasma generator (2) is located. Finally, the thin slat (25) made of metallic material placed horizontally at the outlet can guide the air flow to project it towards a particular direction.

Thus, the air (24) is absorbed by the inlet (20) of the respiratory device (1) due to the depression caused inside the box (19) by the air impeller (18), forcing the incoming flow to pass through the HEPA filter. The air is projected forward forming a laminar flow through the rectangular nozzle (22) wherein part of it is injected into the cold atmospheric plasma generator (2). Finally, the main air flow and the air flow activated by the cold atmospheric plasma generator (2) mix at the outlet opening (23) of the respiratory device (1) projecting onto the patient (5) for inhalation.

The cold atmospheric plasma generator (2) has been built with electrodes (6) and (7) made of brass with a diameter of 10 mm in the space (15) for the formation of the plasma, wherein the inter-electrode separation is 8 mm. The supporting piece of insulating material (10) is made of acetyl resin while the one that supports the flat metallic electrode (7) is made of aluminum. Both have a diameter of 39.9 mm at the height of their respective inserts with O-rings (11). The tube of insulating material is made of quartz with a length of 50 mm and an internal diameter of 40 mm having a wall thickness of 2 mm. The flat metallic electrode (7) has 12 through holes (14) of 1 mm diameter each one distributed in a diameter of 8 mm around its longitudinal axis. A very important aspect regarding the security of the system is that the design of the flat metallic electrode (7) has the advantage of not allowing access to the space (15) for creating the plasma accidentally or intentionally. The small diameter (1 mm) of its through holes (14) does not allow access to the plasma zone, avoiding any risk of electrocution.

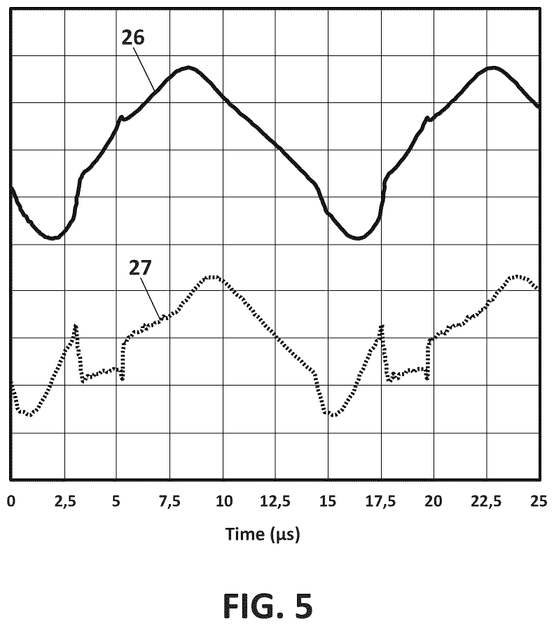

The cold atmospheric plasma generator (2) is powered by a pulsed high tension source at a frequency of 60 kHz which, without load (no load), produces a sinusoidal output tension of 10 kV peak to peak. When the dielectric strength of the air between the electrodes (6), (7) is exceeded, a column of cold atmospheric plasma is established that conducts electricity with a maximum peak current of 60 mA and a tension that is established at 1.82 kV. This working situation, when the cold atmospheric plasma is established, is shown in FIG. 5 wherein the tension (26) and current (27) signals measured with an oscilloscope are observed. These curves provide the electrical characterization of the used plasma.

Measurements were made with an oscilloscope and tension and current probes, obtaining values of 60 kHz frequency, 1.82 kV peak-to-peak tension and 60 mA peak current.

In addition, spectroscopic measurements were made in the ultraviolet-visible and ultraviolet range of the light emitted by the plasma. These measurements are very important to check the existence of RONS in the plasma being used and their relative abundance with respect to non-reactive species in air plasmas such as emissions due to single excited neutral atoms and/or molecules. For this, two fiber optic spectrometers were used whose wavelength range is between 200 to 800 nm (UV and Visible) and 200 to 400 nm (UV). The first is used to see the full spectrum and detect emissions that include, in addition to those from RONS, those from neutral atoms and molecules. The second is of higher resolution and allows obtaining emissions only in the UV range where those due to reactive oxygen and nitrogen molecules, RONS, which are of interest for this patent, are found.

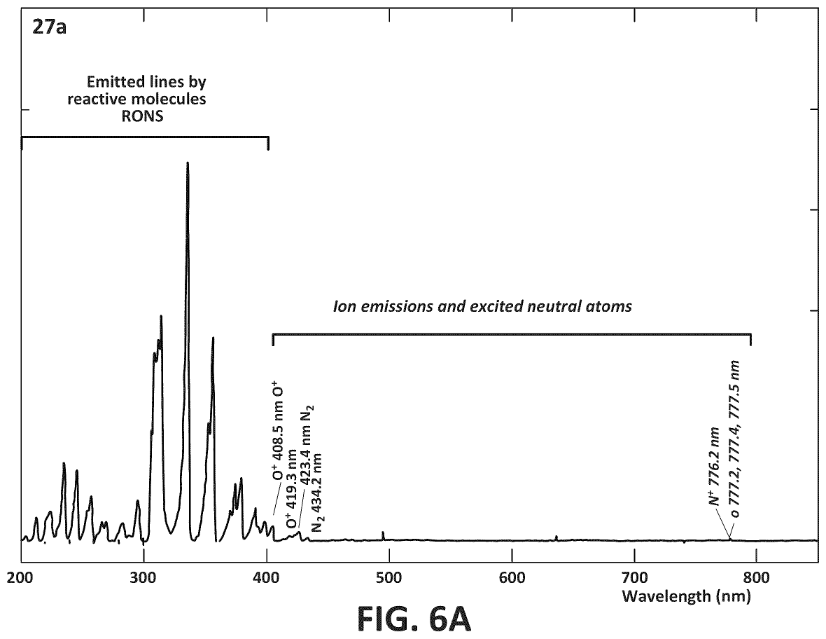

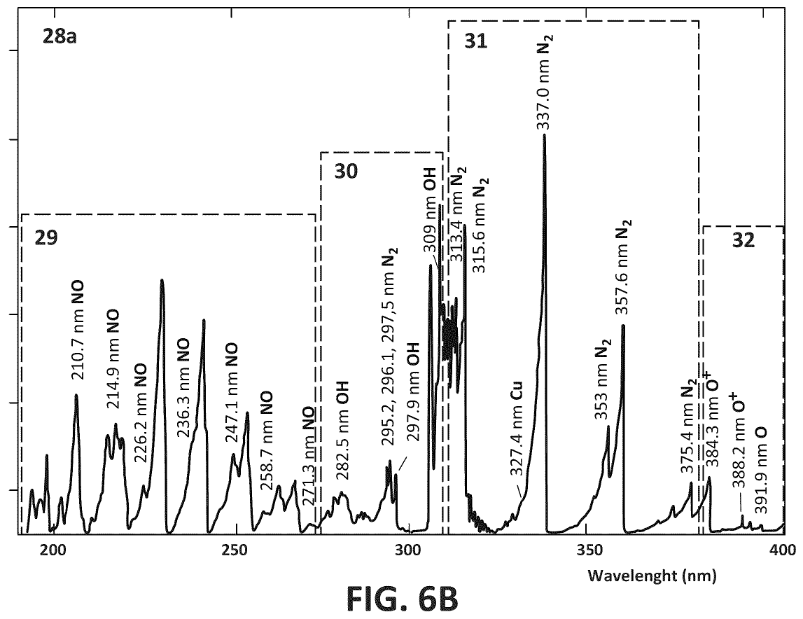

FIGS. 6A and 6B show the result of the spectral measurements wherein in the image (27a) the complete spectrum is observed in the wavelength range 200 to 800 nm corresponding to UV-VIS. In this spectrum it can be seen that the emission in the visible spectrum (400 to 800 nm) is very low compared to the emission in the ultraviolet range (200 to 400 nm). This abundance in the ultraviolet region implies a relatively high production of RONS. A more detailed spectrum of the UV range is shown in the lower image (28) of FIG. 6 wherein the emission between 200 to 400 nm is measured.

The spectrum shows the emission in the wavelength range 200 to 800 nm corresponding to ultraviolet and visible, UV-VIS. In FIG. 6B, image (28a) shows the spectrum with emission only in the ultraviolet range, UV, wherein the spectral lines of NO (nitric oxide) (29), OH⁻(hydroxide ion) (30), N₂ (molecular nitrogen) (31) and neutral and ionized atomic oxygen (32).

The emission of abundant spectral lines is clearly observed, which can be classified into four differential characteristic groups, a first group in the range from 200 to 275 nm corresponding to the emissions of the reactive molecule of nitric oxide NO, a second group in the range from 275 to 310 nm corresponding to the emissions of the reactive molecule of the hydroxide ion OH⁻, a third group of emissions in the range from 310 to 380 nm from the excited neutral molecule of nitrogen N₂ and finally a group of low intensity emissions in the range from 380 to 400 nm from excited neutral atoms and oxygen ions. The cold atmospheric plasma generator has been optimized both in its construction parameters and in those of its electrical supply for the production of RONS, as shown by the measurements carried out. The light radiation analyzed was obtained from inside the flat metallic electrode (7) by introducing an optical fiber from the outlet (17). This allowed the training of light coming from the plasma through the through holes (14).

1. Cold atmospheric plasma generator configured to receive air as input substance and expel RONS, Reactive Oxygen and Nitrogen Species, suitable for being inhaled by living beings, as output substance comprising:
a tube of insulating material defining a cavity;
a supporting piece of insulating material coupled to a first end of the tube of insulating material;
a metallic piece coupled to a second end of the tube of insulating material (8);
a frustoconical positive electrode established in the cavity of the tube of insulating material and connectable to a voltage source;
a flat metallic electrode established in the cavity of the tube of insulating material and connectable to ground,
wherein the electrodes face each other defining a space therebetween in a hollow pin-pepper pot configuration;
wherein the supporting piece of insulating material is configured to hold the frustoconical positive electrode,
wherein the metallic piece (13) is configured to hold the flat metallic electrode,
wherein the frustoconical positive electrode has a through axial perforation configured to allow the passage of air into the cavity of the tube of insulating material,
wherein the flat metallic electrode comprises an axial cavity and a plurality of through holes on the surface of the flat metallic electrode that communicate the axial cavity with the cavity of the tube of insulating material,
wherein the cold atmospheric plasma generator is configured to:
establish a circulating air current between the electrodes and;
apply a voltage to the frustoconical positive electrode greater than the dielectric strength of the air in the space between the electrodes and;
generate a column of cold atmospheric plasma from the circulating air between the electrodes, and
expel the RONS generated in the cold atmospheric plasma column through the axial cavity of the flat metallic electrode to the outside of the cold atmospheric plasma generator.

2. The cold atmospheric plasma generator according to claim 1, wherein the electrodes have cylindrical symmetry and are established in a coaxial position by the supporting pieces of insulating material and the metallic piece, respectively.

3. The cold atmospheric plasma generator according to claim 1, wherein the supporting piece of insulating material is coupled to a first end of the tube of insulating material by means of a first O-ring.

4. The cold atmospheric plasma generator according to claim 1, wherein the metallic piece is coupled to a second end of the tube of insulating material by means of a second O-ring.

5. The cold atmospheric plasma generator according to claim 1, wherein the electrodes comprise brass and a diameter of 10 mm and an inter-electrode separation is 8 mm.

6. The cold atmospheric plasma generator according to claim 1, wherein the supporting piece of insulating material is made of acetyl resin and with a diameter of 39.9 mm.

7. The cold atmospheric plasma generator according to claim 1, wherein the metallic piece comprises aluminum and a diameter of 39.9 mm.

8. The cold atmospheric plasma generator according to claim 1, wherein the tube of insulating material is made of quartz with a length of 50 mm, an internal diameter of 40 mm and a wall thickness of 2 mm.

9. The cold atmospheric plasma generator according to claim 1, wherein the flat metallic electrode comprises 12 through holes of 1 mm diameter each one distributed in a diameter of 8 mm around the longitudinal axis thereof.

10. A respiratory device comprising an inlet opening and an outlet opening, the device comprising:
an air impeller configured to draw air from the inlet opening;
an air filter configured to generate filtered air driven by the air impeller;
the plasma generator according to claim 1 configured to absorb part of the filtered air as input substance and expel RONS, Reactive Oxygen and Nitrogen Species, as output substance; and
a nozzle configured to expel the filtered air enriched with RONS through the outlet opening in a laminar flow,
wherein the laminar flow is apt to be inhaled by living beings.

11. The respiratory device according to claim 10, further comprising an airtight box of insulating material that houses the air impeller,
wherein the air impeller comprises cylindrical blades 30 cm long.

12. The respiratory device according to claim 10, wherein the air filter is a HEPA, High-Efficiency Particulate Arrestance filter whose measurements are 30 cm long by 10 cm wide with a thickness of 3 cm.

13. The respiratory device according to claim 10, wherein the nozzle has a rectangular shape and is made of sheet metal.

14. The respiratory device according to claim 10, comprising a slat configured to guide the laminar flow.

15. The respiratory device according to claim 1 the preceding claims, wherein the slat comprises metal.
